(19) United States
(12) Patent Application Publication
(10) Pub. No.: US 2025/0185142 A1
(43) Pub. Date: Jun. 5, 2025

(54) UV-B LIGHTING SYSTEM AND METHOD

(71) Applicant: SIGNIFY HOLDING B.V., EINDHOVEN (NL)

(72) Inventor: TIES VAN BOMMEL, HORST (NL)

(21) Appl. No.: 18/840,539

(22) PCT Filed: Feb. 16, 2023
(86) PCT No.: PCT/EP2023/053884

§ 371 Date: Aug. 22, 2024

(30) Foreign Application Priority Data
Feb. 24, 2022 (EP) 22158593.8

(51) Int. Cl. H05B 47/155; A01K 45/00; H05B 47/115; H05B 47/16

(52) U.S. Cl. CPC H05B 47/155; A01K 45/00; H05B 47/115; H05B 47/16

(57) ABSTRACT

An agricultural UV-B lighting system has first and second UV-B light sources with different dominant peak wavelengths (one above 300 nm and one below 300 nm). In a first mode, light is generated with a first ratio of the two light source output intensities and in a second mode, light is generated with a second, different, ratio of the light source output intensities. The goals of sterilization, Vitamin D production, safety, and simulation of natural lighting conditions can thereby be balanced.

### FIELD OF THE INVENTION

This invention relates to UV-B lighting systems and methods.

### BACKGROUND OF THE INVENTION

Various properties of UV lighting, and in particular UV-B lighting, have been widely researched. For the purposes of this disclosure, the UV-B wavelength range is defined to be 280 nm to 320 nm. This invention is of particular interest to lighting systems for delivering UV light, in particular UV-B light, to animals (e.g. humans or livestock).

It is well known that UV light stimulates the generation of Vitamin D in many animals, including humans as well as various livestock animals. For example, recent research has shown that the Vitamin D content in eggs can be increased by exposing chickens to UV-B light. UV-B exposure applied to laying hens during the later phase of the laying cycle has also been shown to promote health and welfare of laying hens. The UV-B radiation improves bone density, egg production, eggshell quality and yolk concentration.

Sterilizing properties of UV light are also well known. It is well known that UV light can be used for disinfection i.e. inactivating/killing of viruses and/or bacteria.

Finally, while UV light is invisible to humans, different animals have different sensitivity to the wavelengths of the electromagnetic spectrum. This needs to be taken into account when devising a lighting program for example for livestock farming.

There is therefore a need for a UV-B lighting system with improved performance, functionality and/or safety.

### SUMMARY OF THE INVENTION

The invention is defined by the claims.

According to examples in accordance with an aspect of the invention, there is provided a (UV-B) lighting system comprising:
a first UV-B (solid-state) light source emitting, in operation, first UV-B light with a first dominant peak wavelength below 300 nm;
a second UV-B (solid-state) light source emitting, in operation, second UV-B light with a second dominant peak wavelength above 300 nm, wherein the dominant peak wavelengths differ by at least 10 nm; and
a controller for controlling the output intensities delivered by the first and second UV-B (solid-state) light sources, wherein the controller is operable in:
   a first mode to generate a first light output with a first ratio, R1, of a first UV-B (solid-state) light source output intensity to a second UV-B (solid-state) light source output intensity; and
   a second mode to generate a second light output with a second ratio, R2, of the first UV-B (solid-state) light source output intensity to the second UV-B (solid-state) light source output intensity, wherein R1>R2.

Preferably, each of the output intensities delivered by the first and second UV-B (solid-state) light sources is individually controllable, and the controller is provided for individually controlling each of the output intensities of these light sources.

The UV-B lighting system according to the invention provides an improved performance, functionality and/or safety.

It is noted that the term “light” is used to refer generally to the electromagnetic spectrum, and specifically is intended to include the infrared, visible and ultraviolet part of the spectrum. Thus, a “light source” is not intended to be limited to a visible light source.

This lighting system enables control of the amounts of UV-B light in two wavelength bands/ranges. The UV-B light (across the full 280 nm to 320 nm width of the UV-B spectrum) is effective in promoting the generation of Vitamin D in various animals (including humans). However, it appears that the longer wavelength end of the UV-B wavelength range (e.g. 300 nm to 320 nm) is particularly suitable for destroying bacteria and is rather safe, but less suitable for inactivating viruses. It also appears that the shorter wavelength end of UV-B wavelength range (e.g. 280 nm to 300 nm) is, additional to killing bacteria, also particularly suitable for inactivating viruses, but is less safe (at high dose i.e. high intensity and/or exposure times). The dose (i.e. the light intensity times the exposure time) that can be administered safely depends on the wavelength, with longer wavelengths being less harmful than shorter wavelengths. Thus, the wavelength band with a dominant peak wavelength below 300 nm is also particularly effective in destroying bacteria and viruses, but a high dose may not be desired as it is more harmful in creating tissue damage than longer wavelengths. The wavelength band with a dominant peak wavelength above 300 nm is for example effective in destroying bacteria and can be safely applied with a higher intensity.

Furthermore, some livestock (such as birds or chickens) are able to perceive light in the longer UV wavelengths. The shorter wavelength end of the UV-B spectrum, hence the wavelength light output of the first UV-B light source, however remains invisible/unperceived and hence can be provided during the night (e.g. for stimulating Vitamin D production) whereas the first UV-B light source output may be providing, additionally or alternatively, during the day to better simulate the broad light spectrum of natural daylight. Thus, even within the UV-B band, some wavelengths may be visible to or sensed by some animals while others are not.

Furthermore, the allowable dose which can be applied in a safe way depends on the distance of the animal to the light source.

Furthermore, UV-B light sources emitting low wavelength UV-B light appear to be less efficient and/or more expensive.

The lighting system thus enables the performance, functionality and/or safety of the lighting system to be optimized for a particular purpose. The dominant peak wavelengths may differ by at least 15 nm, for example by at least 20 nm or even by at least 25 nm.

The first ratio is for example greater than 1 and the second ratio is for example less than 1 Thus, a different one of the two light source outputs is dominant in each of the two modes.

The first light output may have no second UV-B light source output and/or the second light output may have no first UV-B light source output.

In other words, all of the light output in the first mode is the first light source output (below 300 nm), hence the ratio is infinite, and/or all of the light in the second mode is the second light source output (above 300 nm), hence the ratio is zero. This results in very different lighting quality or effect between the two modes, for example in terms of disinfection performance vs. safety or visibility (e.g. for birds such as poultry).

Instead, the first and second light outputs may each have components from both the first and second UV-B light sources, in different proportions. Thus, the two modes may have different mixes of the two wavelengths. Thus, a balance can be optimized in terms of performance and safety.

A total intensity from the first and second solid state UV-B light sources (i.e. I1+I2) is for example lower in the first mode than in the second mode. It is more safe to provide a higher intensity for the longer wavelength UV-B light of the second mode.

The first dominant peak wavelength is for example below 295 nm especially below 290 nm and/or the second dominant peak wavelength is for example above 305 nm especially above 310 nm. This provides spacing from the threshold wavelength of 300 nm.

The lighting system may further comprise a white light source, wherein the controller is further for controlling the output intensity delivered by the white light source. The output intensity delivered by the white light source may be individually controllable, independent from the output intensities of the first and second UV-B (solid-state) light sources, and the controller may be provided for individually controlling the output intensity of the white light source. The white light source is used to better mimic the light spectrum during the day, for example with a combination of visible and UV-B light. The white light emitted by the white light source may have a correlated color temperature in a range from 2000K to 10000K and/or a color rendering index of at least 70 especially at least 80.

The first light output for example has no white light source output and the second light output has a white light source output component. The first light output is for example for use during darkness and the second light output is for example for use during the day.

The lighting system for example comprises a timer (i.e., a clock module) for operating the controller in the first mode and the second mode according to a time sequence. A user interface may be provided, wherein the timer is programmable via the user interface.

In one example, the time sequence provides the first light output during the night and the second light output during the day. For example, the first light output may be provided at least at one point during the night such as at or around midnight, and the second light output may be provided at least at one point during the day, such as at or around midday. Thus, natural lighting conditions as well as the intended purpose of the UV-B lighting, such as Vitamin D production and/or sterilization, are taken into account when scheduling and/or applying the time sequence.

In embodiments, said UV-B lighting system may comprise a light exit window for exiting the first UV-B light and the second UV-B light, and optionally the white light.

The lighting system for example comprises a livestock farming lighting system for example a bird or poultry farming lighting system.

In embodiments, the lighting system further comprises a sensor configured to detect at least one of a presence and a location of one or more animals and to determine a distance between (i) the one or more animals and (ii) a lighting arrangement comprising the first and second UV-B solid-state light sources, wherein the sensor is functionally coupled to the controller and the controller is further configured to control and adjust a ratio R between the first UV-B solid state light source output intensity and the second UV-B solid state light source output intensity as a function of the determined distance between the one or more animals and the lighting arrangement.

In embodiments, the controller is configured to decrease the ratio when the determined distance is below a predefined threshold (e.g. <3 m) and to increase the ratio when the determined distance is above the predefined threshold (e.g. >3 m).

In embodiments, the controller is arranged to decrease the ratio by increasing the intensity of the second UV-B solid-state light source output intensity, and to increase the ratio by decreasing the intensity of the second UV-B solid-state light source output intensity.

In embodiments, the controller is arranged to decrease the ratio by decreasing the intensity of the first UV-B solid-state light source output intensity, and to increase the ratio by increasing the intensity of the first UV-B solid-state light source output intensity.

In embodiments, the controller is arranged to decrease the ratio by increasing the intensity of the second UV-B solid-state light source output intensity and decreasing the intensity of the first UV-B solid-state light source output intensity, and to increase the ratio by decreasing the intensity of the second UV-B solid-state light source output intensity and by increasing the intensity of the first UV-B solid-state light source output intensity.

In embodiments, when the ratio is decreased the total intensity (i.e. the first and second UV-B solid-state light source output intensity) is increased and when the ratio is increased the total intensity (i.e. the first and second UV-B solid-state light source output intensity) is decreased.

In embodiments, the UV-B solid-state light source output intensity is zero if the one or more animals are within the predefined threshold (e.g. <3 m) for the determined distance between the one or more animals and the lighting arrangement (i.e. the first and second UV-B solid-state light source).

The invention also provides a method of controlling a UV-B lighting system, the lighting system comprising a first UV-B solid state light source emitting, in operation, a first dominant peak wavelength below 300 nm and a second solid state UV-B light source emitting, in operation, a second dominant peak wavelength above 300 nm, wherein the dominant peak wavelengths differ by at least 10 nm, wherein the method comprises:
controlling the output intensities delivered by the first and second solid state UV-B light sources such that:
at a first time a first light output is generated with a first ratio, R1, of the first UV-B solid state light source output intensity to the second UV-B solid state light source output intensity; and
at a second time a second light output is generated with a second ratio, R2, of the first UV-B solid state light source output intensity to the second UV-B solid state light source output intensity, wherein wherein R1>R2.

The first time is for example during the night (or other resting time of livestock) and then the first light source output is dominant. The second time is for example during the day and then the second light source output is dominant.

The method may comprise providing the first and second light outputs to livestock of a livestock farming system, such as to poultry of a poultry farming system.

The invention also provides a computer program comprising computer program code means which is adapted, when said program is run on a computer, to implement the method defined above.

The disclosed (UV-B) lighting system and method of controlling such (UV-B) lighting system are especially suitable for use in agriculture applications such as livestock farming (e.g., poultry farming) and the (UV-B) lighting system may be configured to provide illumination to livestock (e.g., poultry) in a farm. The disclosed (UV-B) lighting system may therefore also be referred to as an agriculture (UV-B) lighting system such as a livestock farming (UV-B) lighting system (e.g., a poultry farming (UV-B lighting system)). For example, the disclosed (UV-B) lighting system may be configured to comply with certain ingress protection rating (IP Rating) requirements for agriculture environment such as livestock farming environments (e.g., poultry farming environments). Some disclosed (UV-B) lighting systems may be configured for high bay application in agriculture such as livestock farming (e.g., poultry farming).

These and other aspects of the invention will be apparent from and elucidated with reference to the embodiment(s) described hereinafter.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 shows a lighting system;

FIG. 2 shows sensitivity to light wavelengths; and

FIG. 3 shows a lighting control method.

### DETAILED DESCRIPTION OF THE EMBODIMENTS

The invention will be described with reference to the Figures.

It should be understood that the detailed description and specific examples, while indicating exemplary embodiments of the apparatus, systems and methods, are intended for purposes of illustration only and are not intended to limit the scope of the invention. These and other features, aspects, and advantages of the apparatus, systems and methods of the present invention will become better understood from the following description, appended claims, and accompanying drawings. It should be understood that the Figures are merely schematic and are not drawn to scale. It should also be understood that the same reference numerals are used throughout the Figures to indicate the same or similar parts.

The invention provides a UV-B lighting system which has first and second UV-B light sources with different dominant peak wavelengths (one above 300 nm and one below 300 nm). In a first mode, light is generated with a first ratio of the two light source output intensities and in a second mode, light is generated with a second, different, ratio of the light source output intensities. The goals of sterilization, Vitamin D production, safety, and simulation of natural lighting conditions can thereby be balanced.

The invention makes use of solid state light sources. These will however simply each be referred to as a “light source” in the description below.

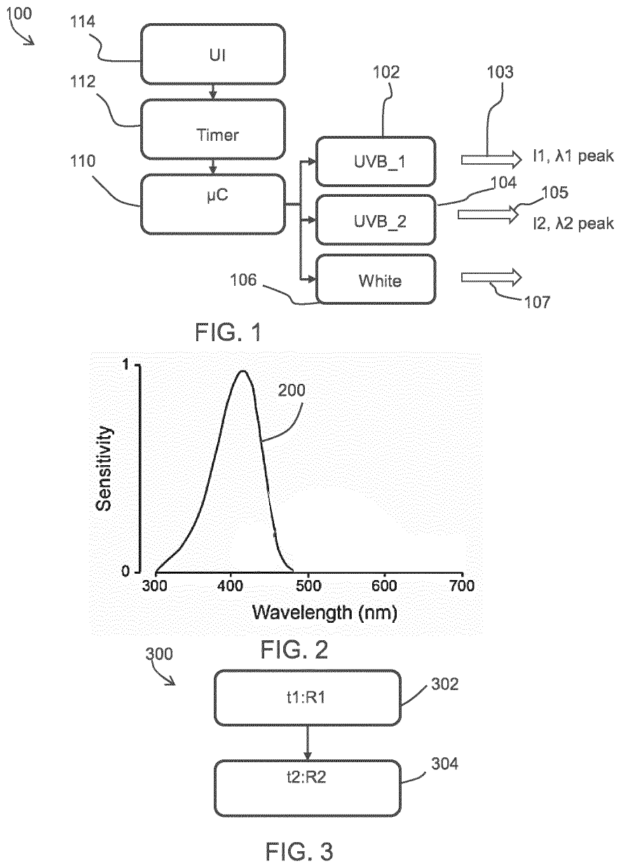

FIG. 1 shows UV-B lighting system 100 comprising a first UV-B light source 102 with a first dominant peak wavelength 21 (i.e. wavelength at peak intensity) below 300 nm and a second UV-B light source 104 with a second dominant peak wavelength 22 above 300 nm. The first UV-B light source 102 delivers a light output 103 with an output intensity I1 and the second UV-B light source 104 delivers a light output 105 with an output intensity I2. These intensities are controllable at least between on and off, and optionally also between analogue intensity levels. Preferably, each of these intensities is individually controllable. The total UV-B intensity is thus I1+I2.

In a preferred example, the first dominant peak wavelength is below 290 nm and the second dominant peak wavelength is above 310 nm.

The dominant peak wavelength is the wavelength at which the light output intensity is highest. In practice, each light source will generate a band of wavelengths, with the dominant peak wavelength near the center of the band. These bands may overlap. However, the dominant peak wavelengths differ by at least 10 nm, preferably at least 15 nm, more preferably at least 20 nm, most preferably at least 25 nm. A larger difference may be better in terms of disinfection performance vs. safety or visibility (e.g. for poultry).

A controller 110 is provided for controlling the output intensities delivered by the first and second UV-B light sources 102, 104. Preferably, the controller is provided for individually controlling each of the output intensities of the light sources. In particular, a different combination of the two wavelength bands may be provided at different times, to achieve different lighting aims.

There may be multiple combinations of different light intensities from the two light sources 102, 104, giving multiple operating modes. However, as a minimum, there are at least two modes.

In the first mode, a first light output is generated with a first ratio R1 of the first UV-B light source output intensity to the second UV-B light source output intensity. This ratio R1 is for example greater than 1, meaning that the output intensity of the first light source (below 300 nm) is dominant.

In the second mode, a second light output is generated with a second, different, ratio R2 of the first UV-B light source output intensity to the second UV-B light source output intensity, This ratio R2 is for example less than 1, meaning that the output intensity of the second light source (above 300 nm) is dominant.

In one example, only one of the two light sources is turned on at any time. Thus, the first light output has no second UV-B light source output (R1=∞) and the second light output has no first UV-B light source output (R2=0).

Instead, both light sources may be on for one or both of the modes so that 1<R1<∞ for the first light output and 0<R2<1 for the second light output.

A total intensity from the first and second solid state UV-B light sources (i.e. I1+I2) may be controlled to be lower in the first mode than in the second mode. It is more safe to provide a higher intensity for the longer wavelength UV-B light of the second mode.

FIG. 1 also shows a white light source 106 controlled by the controller for delivering a white light output 107.

The system thus enables control of the amounts of UV-B light in two wavelength bands to be controlled. The particular characteristics of the light output at any time are chosen to achieve one or more of several objectives:

Vitamin D production;

Bacteria sterilization;

Virus sterilization;

Mimicking natural daylight (as perceived by a target animal species);

Mimicking natural night light (as perceived by a particular target animal species, e.g. avoiding disturbance during animal rest);

Ensuring a safe UV dose.

By way of example, it has been shown that chickens can perceive light down to wavelengths of around 300 nm. Thus, to avoid disturbance during the night or rest times, the UV-B light above 300 nm is not desired. However, during the day, this part of the spectrum is present in sunlight so is desirable to better mimic the broad spectrum of natural sunlight.

The first light output (with dominant peak wavelength below 300 nm) for example has no white light source output component since this may be used during darkness, whereas the second light output (with dominant peak wavelength above 300 nm) has a white light source output component.

Thus, the first light output has no white light and R1>1 whereas the second light output has white light and R2<1.

Thus, it can be seen that the two or more modes may be applied as a function of the time of day. For this purpose, FIG. 1 shows a timer 112 (i.e., clock) for operating the controller in the first mode and the second mode in accordance with a time sequence. A user interface 114 is for example used to enable a user of the system to program the timer. Thus user interface may be implemented by an app loaded onto a mobile phone. The program determines a value of R, such as R1 and R2 referred to above, for different time slots as well as the white light intensity (when the optional white light source is used).

The white light may have an intensity and/or color properties (e.g., color temperature) that varies over time, again to mimic natural lighting conditions such as dawn and sunset. The white light source may be individually controllable independent from the first UV-B light source and the second UV-B light source.

FIG. 2 shows a plot of the sensitivity to light in the wavelength range 300 nm to 700 nm for chickens. It shows that the sensitivity starts at 300 nm.

The control of the UV-B light may also take account of the distance between the lighting arrangement (comprising the first and second UV-B solid-state light sources) and animals in an enclosure. In this way, the dose of the different light delivered to the animals may be controlled, also taking account of the different attenuation for different wavelengths. For this purpose, a sensor may be provided to detect at least one of a presence and a location of one or more animals and to determine a distance between (i) the one or more animals and (ii) the lighting arrangement.

The controller may then control and adjust a ratio between the first UV-B solid state light source output intensity and the second UV-B solid state light source output intensity as a function of the determined distance between the one or more animals and the lighting arrangement.

The ratio is for example decreased (so relatively less of the first UV-B light) when the animals are close, i.e., the determined distance is below a predefined threshold, and increased (so relatively more of the first UV-B light) when the determined distance is above the predefined threshold.

The threshold is for example 3 m. The ratio may be decreased by increasing the intensity of the second UV-B solid-state light source output intensity, and the ratio may be increased by decreasing the intensity of the second UV-B solid-state light source output intensity.

Alternatively, the ratio may be decreased by decreasing the intensity of the first UV-B solid-state light source output intensity, and the ratio may be increased by increasing the intensity of the first UV-B solid-state light source output intensity.

Alternatively, the ratio may be decreased by increasing the intensity of the second UV-B solid-state light source output intensity and decreasing the intensity of the first

UV-B solid-state light source output intensity, and the ratio may be increased by decreasing the intensity of the second UV-B solid-state light source output intensity and by increasing the intensity of the first UV-B solid-state light source output intensity.

When the ratio is decreased, the total intensity may be increased and when the ratio is increased the total intensity may be decreased.

The UV-B solid-state light source output intensity may be made zero if the one or more animals are within the (or another) predefined distance threshold.

FIG. 3 shows a method 300 of controlling the UV-B lighting system as described above. The method comprises controlling the output intensities delivered by the first and second UV-B light sources such that:
in step 302, at a first time, a first light output is generated with a first ratio R1 of the first UV-B light source output intensity to the second UV-B light source output intensity; and
in step 304, at a second time, a second light output is generated with a second, different, ratio R2 of the first UV-B light source output intensity to the second UV-B light source output intensity.

The control of the light sources is for example implemented by software running on the controller.

The switching between modes may take place between day and night or it may take place with greater frequency, for example such that the daytime is divided into different time zones (e.g. resting and active times). Indeed, a program of values of R for a ratio of the first UV-B light source output intensity to the second UV-B light source output intensity may be divided into time slots of any desired resolution (minutes or hours). For example, the time periods during which the ratio is set at a given value may last at least 1 minute, more preferably at least 10 minutes, and most preferably at least 1 hour. The time periods are for example less than 22 hours, more preferably less than 20 hours, most preferably less than 18 hours. There may for example be a gradual change in the ratio over time.

The light sources for example comprise UV-LEDs and/or UV laser diodes.

The lighting system may include additional light sources to those shown, such as UV-A light sources. However, the first light output and/or the second light output may for example not comprise UV-A light (in the range 320 nm to 400 nm) and/or UV-C light (in the range 100 nm to 280 nm).

Furthermore, the lighting system may be combined with a ventilation and/or heating system to provide an overall system for controlling the environmental conditions, for example of farming livestock.

Sensors may be provided for automating or partially automating the control of the lighting system. The lighting modes may be selected based on sensed conditions (e.g. activity levels) of the livestock.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality.

Functions implemented by a processor may be implemented by a single processor or by multiple separate processing units which may together be considered to constitute a “processor”. Such processing units may in some cases be remote from each other and communicate with each other in a wired or wireless manner.

The mere fact that certain measures are recited in mutually different dependent claims does not indicate that a combination of these measures cannot be used to advantage.

A computer program may be stored/distributed on a suitable medium, such as an optical storage medium or a solid-state medium supplied together with or as part of other hardware, but may also be distributed in other forms, such as via the Internet or other wired or wireless telecommunication systems.

If the term “adapted to” is used in the claims or description, it is noted the term “adapted to” is intended to be equivalent to the term “configured to”. If the term “arrangement” is used in the claims or description, it is noted the term “arrangement” is intended to be equivalent to the term “system”, and vice versa.

Any reference signs in the claims should not be construed as limiting the scope.

1. A livestock farming UV-B lighting system comprising:
a first UV-B solid-state light source emitting, in operation, first UV-B light with a first dominant peak wavelength below 300 nm;
a second UV-B solid-state light source emitting, in operation, second UV-B light with a second dominant peak wavelength above 300 nm, wherein the dominant peak wavelengths differ by at least 10 nm; and
a controller for individually controlling the output intensities delivered by the first and second UV-B solid-state light sources, wherein the controller is operable in:
a first mode to generate a first light output with a first ratio, R1, of a first UV-B solid-state light source output intensity to a second UV-B solid-state light source output intensity; and
a second mode to generate a second light output with a second ratio, R2, of the first UV-B solid-state light source output intensity to the second UV-B solid-state light source output intensity, wherein R1>R2.

2. The livestock farming lighting system of claim 1, wherein the first ratio, R1, is greater than 1 and the second ratio, R2, is less than 1.

3. The livestock farming lighting system of claim 1, wherein:
the first light output has no second UV-B solid-state light source output; and/or
the second light output has no first UV-B solid-state light source output.

4. The livestock farming lighting system of claim 1, wherein the first and second light outputs each have components from both the first and second UV-B solid state light sources, in different proportions.

5. The livestock farming lighting system of claim 1, wherein a total intensity from the first and second solid state UV-B light sources is lower in the first mode than in the second mode.

6. The livestock farming lighting system of claim 1, wherein the first dominant peak wavelength is below 290 nm and/or the second dominant peak wavelength is above 310 nm.

7. The livestock farming lighting system of claim 1, further comprising a white solid-state light source emitting, in operation, white light, wherein the controller is further configured for controlling the output intensity delivered by the white solid-state light source.

8. The livestock farming lighting system of claim 7, wherein the first light output has no white light and the second light output has white light.

9. The livestock farming lighting system of claim 1, comprising a timer configured to operate the controller in the first mode and the second mode according to a time sequence; and a user interface, wherein the timer is configured to be programmable with the time sequence via the user interface.

10. (canceled)

11. The livestock farming lighting system of claim 9, wherein the time sequence provides the first light output during the night and the second light output during the day.

12. The livestock farming lighting system according to claim 1, further comprising a sensor configured to detect at least one of a presence and a location of one or more animals and to determine a distance between (i) the one or more animals and (ii) a lighting arrangement comprising the first and second UV-B solid-state light sources, wherein the controller is further configured to control and adjust a ratio R between the first UV-B solid state light source output intensity and the second UV-B solid state light source output intensity as a function of the determined distance between the one or more animals and the lighting arrangement.

13. The livestock farming lighting system of claim 12, wherein the controller is configured to decrease a ratio R between the first UV-B solid state light source output intensity and the second UV-B solid state light source output intensity when the determined distance is below a predefined threshold and to increase the ratio R when the determined distance is above the predefined threshold

14. The livestock farming lighting system of claim 1, wherein the livestock farming lighting system is, a poultry farming lighting system.

15. A method of controlling a livestock farming UV-B lighting system, the lighting system comprising a first UV-B solid state light source emitting, in operation, a first dominant peak wavelength below 300 nm and a second solid state UV-B light source emitting, in operation, a second dominant peak wavelength above 300 nm, wherein the dominant peak wavelengths differ by at least 10 nm, wherein the method comprises:
Individually controlling the output intensities delivered by the first and second solid state UV-B light sources such that:
at a first time a first light output is generated with a first ratio, R1, of the first UV-B solid state light source output intensity to the second UV-B solid state light source output intensity; and
at a second time a second light output is generated with a second ratio, R2, of the first UV-B solid state light source output intensity to the second UV-B solid state light source output intensity, wherein R1>R2.

16. The method of claim 15, further comprising controlling the output intensities delivered by the first and second solid state UV-B light sources such that the first time occurs during a night or resting time of a livestock and the second time occurs during the day of the livestock.
